SEQUENCE LISTING (14 sequences)

SEQ ID NO: 1 - DNA, 18 bp, synthetic construct
  source 1..18: mol_type = other DNA
tcctccggagccagactt

SEQ ID NO: 2 - DNA, 17 bp, synthetic construct
  source 1..17: mol_type = other DNA
ttctccactcgtcggcc

SEQ ID NO: 3 - DNA, 18 bp, synthetic construct
  source 1..18: mol_type = other DNA
acaggccgtgtcgttgtc

SEQ ID NO: 4 - DNA, 18 bp, synthetic construct
  source 1..18: mol_type = other DNA
gcactcgccgtcgtggat

SEQ ID NO: 5 - DNA, 18 bp, synthetic construct
  source 1..18: mol_type = other DNA
cggatatggtcgttctcc

SEQ ID NO: 6 - DNA, 18 bp, synthetic construct
  source 1..18: mol_type = other DNA
tctcagcctcgtggttgc

SEQ ID NO: 7 - DNA, 18 bp, synthetic construct
  source 1..18: mol_type = other DNA
ttgcggcctcgttcactg

SEQ ID NO: 8 - DNA, 18 bp, synthetic construct
  source 1..18: mol_type = other DNA
aagcttcgttgagaaact

SEQ ID NO: 9 - DNA, 18 bp, synthetic construct
  source 1..18: mol_type = other DNA
ggacttgctcgttggaca

SEQ ID NO: 10 - DNA, 18 bp, synthetic construct
  source 1..18: mol_type = other DNA
ggctgtctctcgtcgaag

SEQ ID NO: 11 - DNA, 20 bp, synthetic construct
  source 1..20: mol_type = other DNA
cagatttctccactcgtcgg

SEQ ID NO: 12 - DNA, 17 bp, synthetic construct
  source 1..17: mol_type = other DNA
ccggagccagacttcat

SEQ ID NO: 13 - DNA, 20 bp, synthetic construct
  source 1..20: mol_type = other DNA
ctgctcctcctctaggatga

SEQ ID NO: 14 - DNA, 15 bp, synthetic construct
  source 1..15: mol_type = other DNA
ccctcctccggagcc

(19) United States
(12) Patent Application Publication
(10) Pub. No.: US 2026/0364155 A1
(43) Pub. Date: Nov. 19, 2026

(54) METHODS AND COMPOSITIONS FOR TREATING BLADDER CANCER

(71) Applicants: Thomas Jefferson University, Philadelphia, PA (US); ImVAX, Inc., Philadelphia, PA (US)

(72) Inventors: David Andrews, Philadelphia, PA (US); Mark Adrian Exley, Chestnut Hill, MA (US)

(21) Appl. No.: 19/118,505

(22) PCT Filed: Oct. 5, 2023
(86) PCT No.: PCT/US2023/076063

§ 371 Date: Apr. 4, 2025

Related U.S. Application Data
(60) Provisional application No. 63/378,437, filed on Oct. 5, 2022.

(51) Int. Cl. A61K 39/00; A61K 35/12; A61K 35/13; A61M 37/00; A61P 35/00; C12N 5/00; C12N 5/09; C12N 13/00; C12N 15/113

(52) U.S. Cl. CPC A61K 39/0011; A61K 35/13; A61M 37/0069; A61P 35/00; C12N 5/0012; C12N 5/0018; C12N 5/0693; C12N 13/00; C12N 15/1138; A61K 2035/126; A61K 2039/5152; C12N 2310/11; C12N 2501/105

(57) ABSTRACT

The present disclosure relates to compositions and methods for treating bladder cancer. In some embodiments the compositions and methods involve using antisense (AS) nucleic acids directed against Insulin-like Growth Factor 1 Receptor (IGF-1R). The AS may be administered to the patients systemically, or may be used to produce an autologous cancer cell vaccine. In embodiments, the AS are provided in an implantable irradiated biodiffusion chamber comprising tumor cells and an effective amount of the AS. The chambers are irradiated and implanted in the abdomen of subjects and stimulate an immune response that attacks tumors distally. The compositions and methods disclosed herein may be used to treat many different kinds of bladder cancer, including metastatic breast cancer.

### CROSS-REFERENCE TO RELATED APPLICATIONS

This application claims priority to U.S. Provisional Application Ser. No. 63/378,437, filed Oct. 5, 2022, the disclosures of which is hereby incorporated by reference herein in its entirety.

### FIELD

The present disclosure relates to compositions and methods for treating bladder cancer.

### DESCRIPTION OF THE TEXT FILE SUBMITTED ELECTRONICALLY

The contents of the XML file submitted electronically herewith are incorporated by reference in their entirety: a computer readable format copy of the Sequence Listing (filename: 205961_5016_00WO_SequenceListing.xml, date recorded: Oct. 5, 2023, file size 13,436 bytes).

### BACKGROUND

PCT application No. WO2018165528 discloses “compositions and methods for treating cancers using antisense (AS) nucleic acids directed against Insulin-like Growth Factor 1 Receptor (IGF-1R). The AS may be administered to the patients systemically, or may be used to produce an autologous cancer cell vaccine. In embodiments, the AS are provided in an implantable irradiated biodiffusion chamber comprising tumor cells and an effective amount of the AS.”

### SUMMARY

The present disclosure is based, at least in part, on the finding that a subject having bladder cancer can be vaccinated against the cancer, for example, by harvesting cells from tumor tissue from the subject, encapsulating the harvested cells in a bio diffusion chamber, irradiating the chamber with the harvested cells, implanting the chamber in the subject, wherein an immune response against the bladder cancer is obtained.

The instant disclosure is also based, at least in part, on the discovery that an antisense oligodeoxynucleotide (AS-ODN) targeting the insulin-like growth factor receptor-1 (IGF-1R) effectively stimulates a response in a subject that treats bladder cancer when used in the therapeutic approaches described herein. In some aspects, methods and compositions are effective for treating bladder cancer in a patient as part of an autologous cancer cell vaccine alone or, optionally, along with systemic administration. In some approaches, the methods disclosed herein provide effective bladder cancer therapy as a monotherapy; i.e. in the absence of chemotherapy and in the absence of radiation therapy.

Accordingly, in one aspect, the present disclosure provides a method of treating a subject suffering from bladder cancer, the method comprising implanting one or more biodiffusion chambers into the subject, wherein the one or more biodiffusion chambers comprise irradiated tumor cells from the subject. In some aspects, and embodiments the present disclosure provides a method of treating a subject suffering from bladder cancer, the method comprising implanting one or more biodiffusion chambers into the subject, wherein the one or more biodiffusion chambers comprise irradiated tumor cells from the subject, and irradiated insulin-like growth factor receptor-1 antisense oligodeoxynucleotide (IGF-1R AS ODN).

In one aspect, provided is a method for preparing a biodiffusion chamber for implantation into a subject having bladder cancer, the method comprising (a) encapsulating tumor cells obtained from the subject into the biodiffusion chamber; and (b) irradiating the biodiffusion chamber. In one embodiment, provided is a method for preparing a biodiffusion chamber for implantation into a subject having bladder cancer, the method comprising (a) encapsulating tumor cells obtained from the subject into the biodiffusion chamber in the presence of an IGF-1R AS ODN; and (b) irradiating the biodiffusion chamber.

In another aspect, a method of vaccinating a subject having bladder cancer is provided where the method includes: (i) obtaining morselized bladder tumor tissue from the subject; (iii) harvesting adherent cells from the tissue; (iv) encapsulating the harvested cells in a biodiffusion chamber; (v) irradiating the chamber, and (vi) implanting the chamber in the subject. In some embodiments, a method of vaccinating a subject having bladder cancer is provided where the method includes: (i) obtaining bladder tumor tissue from the subject; (ii) collecting the morselized tissue in a sterile trap; (iii) harvesting adherent cells from the morselized tissue; (iv) encapsulating the harvested cells in a biodiffusion chamber along with insulin-like growth factor receptor-1 antisense oligodeoxynucleotide (IGF-1R AS ODN) having the sequence of SEQ ID NO:1; (v) irradiating the chamber, and (vi) implanting the chamber in the subject. In some embodiments, the method of vaccinating a subject results in an immune response against the bladder cancer.

In some aspects, the present disclosure provides a biodiffusion chamber for implantation into a subject suffering from bladder cancer, wherein the biodiffusion chamber comprises irradiated bladder tumor cells obtained from the subject's tumor tissue. In certain embodiments, the present disclosure provides a biodiffusion chamber for implantation into a subject suffering from bladder cancer, the biodiffusion chamber comprising irradiated bladder tumor cells from the subject's tumor tissue and irradiated insulin-like growth factor receptor-1 antisense oligodeoxynucleotide (IGF-1R AS ODN).

In many preferred embodiments, the biodiffusion chamber comprises a membrane that is impermeable to the cells and permeable to oligonucleotides such as the IGF-1R AS ODN. In embodiments, the tumor cells are removed from a resection site of the subject. In certain embodiments, the present disclosure provides a diffusion chamber comprising irradiated IGF-1R AS ODN and irradiated, adhesion-enriched, morselized tumor cells; wherein the biodiffusion chamber comprises a membrane that is impermeable to the cells and permeable to the IGF-1R AS ODN.

In some embodiments the tumor cells are removed from the bladder of a subject surgically. In some embodiments the tumor cells are removed from the bladder of a subject via biopsy. In various embodiments, the tumor cells may be removed from the resection site using an endoscopic device.

In some embodiments, the tumor cells are removed from the resection site using a tissue morselator. In other embodiments, the tissue morselator comprises a high-speed reciprocating inner cannula within a stationary outer cannula. The outer cannula may comprise a side aperture, wherein the tumor cells are drawn into the side aperture by electronically controlled variable suction. In embodiments, the tissue morselator does not produce heat at the resection site. In still further embodiments, the tumor cells are enriched for nestin expression before they are placed into the biodiffusion chamber. In some embodiments, implantation of the chamber inhibits regrowth of the tumor in the subject. In some embodiments, implantation of the chamber inhibits regrowth of the tumor for at least 3 months, at least 6 months, at least 12 months, or at least 36 months.

As used herein “irradiate” means exposing cells and/or chambers to a specified amount of either gamma rays, x-rays, or ultraviolet B rays. In some embodiments, irradiation involves exposing cells and/or chambers to gamma rays. In certain aspects, the dose of radiation is between 1Gy and 15 Gy, or between 1 Gy and 5 Gy, or between 5 Gy and 10 Gy, or between 5 Gy and 15 Gy, or about 1 Gy, or about 2 Gy, or about 4 Gy, or about 5 Gy, or about 6 Gy, or about 10 Gy, or up to about 15 Gy. In certain aspects, the dose of radiation is not more than about 5 Gy. In other aspects, the dose of radiation is at least about 5 Gy. In certain aspects, the tumor cells are treated with gamma irradiation at an amount of about 1 Gy to about 200 Gy, for example about 10 Gy, about 20, about 30 Gy, about 40, about 50, about 60 Gy, about 70 Gy, about 80 Gy, about 90 Gy, about 100 Gy, about 125 Gy, about 150 Gy, about 175 Gy, or about 200 Gy, including all values and subranges that lie therebetween.

In additional embodiments, the present disclosure provides a method for preparing a biodiffusion chamber for implantation into a subject suffering from bladder cancer, the method comprising placing tumor cells into the biodiffusion chamber in the presence of an IGF-1R AS ODN, and irradiating the biodiffusion chamber, wherein the tumor cells are removed from a resection site in the subject using a tissue morselator that does not produce heat at the resection site. In some embodiments, the tissue morselator is an endoscopic device. In further embodiments, the tissue morselator comprises a high-speed reciprocating inner cannula within a stationary outer cannula. In additional embodiments, the outer cannula comprises a side aperture, and the tumor cells are drawn into the side aperture by electronically controlled variable suction.

Typically, multiple chambers are used in some embodiments of the disclosure. For example, about 10 chambers, or about 20 chambers may be used. Advantageously, an optimal anti-tumor response is obtained when the number of cells in the chamber is about 750,000 to about 1,250,000; for example about 1,000,000 per chamber where 20 chambers are implanted.

In some of the aspects and embodiments of any of the compositions or methods provided herein, the bladder cancer is a locally advanced or metastatic urothelial cancer (mUC). In some of the aspects and embodiments of any of the compositions or methods provided herein, the bladder cancer is muscle invasive urothelial carcinoma. In some of the aspects and embodiments of any of the compositions or methods provided herein, the bladder cancer is non-muscle invasive urothelial carcinoma. In some of the aspects and embodiments of any of the compositions or methods provided herein, the bladder cancer is Bacillus Calmette-Guerin (BCG)-unresponsive, high-risk, non-muscle invasive bladder cancer (NMIBC) with carcinoma in situ (CIS) with or without papillary tumors in a patient is ineligible for or has elected not to undergo cystectomy. In some of the aspects and embodiments of any of the compositions or methods provided herein, the bladder cancer is upper tract urothelial cancer. In some of the aspects and embodiments of any of the compositions or methods provided herein, the bladder cancer is low grade upper tract urothelial cancer (LG-UTUC).

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts a representative timeline of the Example 1 experiments.

FIG. 2 provides representative summary information of the study design of the Example 1 experiments.

FIG. 3 shows representative survival of MBT-2 challenged mice treated with IUC-001 as compared to mock treated mice in the Example 1 experiments and the improvement in survival associated with IUC-001 treatment.

FIG. 4 shows representative body weight of MBT-2 challenged mice treated with IUC-001 as compared to mock treated mice in the Example 1 experiments.

FIG. 5 shows representative selected clinical observations of MBT-2 challenged mice treated with IUC-001 as compared to mock treated mice in the Example 1 experiments.

FIG. 6 shows representative selected clinical necropsy findings of MBT-2 challenged mice treated with IUC-001 as compared to mock treated mice in the Example 1 experiments.

FIG. 7 shows representative survival of MBT-2 challenged mice treated with IUC-001 as compared to mock treated mice in the Example 1 experiments and the improvement in survival associated with IUC-001 treatment.

FIG. 8 shows representative hunch posture score of MBT-2 challenged mice treated with IUC-001 as compared to mock treated mice and the improvement in hunch posture associated with IUC-001 treatment.

FIG. 9 shows representative distended abdomen data of MBT-2 challenged mice treated with IUC-001 as compared to mock treated mice.

FIG. 10 shows representative end-of-life bladder weights of MBT-2 challenged mice treated with IUC-001 as compared to mock treated mice.

### DETAILED DESCRIPTION

Described herein are compositions and methods for personalized therapy for bladder cancer. More specifically, the disclosure provides a therapeutic approach for bladder cancer that aims at reducing the length and number of post-surgery treatments, especially hormone therapy, thereby also reducing potential side effects. The data presented herein shows that that pre-vaccination with bladder cancer cells treated with the IGF-1R antisense can prevent tumor growth. Accordingly, the disclosure provides a promising new treatment for bladder cancer patients, including those affected by advanced stage or triple-negative cancer. Specifically, the disclosure provides for treatment of bladder cancer patients with a single post-surgery autologous vaccine.

As used herein, terms such as “a,” “an,” and “the” include singular and plural referents unless the context clearly demands otherwise.

As used herein, the term “about” when preceding a numerical value indicates the value plus or minus a range of 10%. For example, “about 100” encompasses and includes 90 and 110, as well as 100.

As used herein, the term “autologous” means cells or tissues obtained from the same individual.

As used herein, the term “autologous cancer cell vaccine” refers to a therapeutic produced in part by isolating tumor cells from an individual and processing these tumor cells ex vivo. The cells, or cells derived therefrom, are then re-administered to the individual from whom the tumor cells were isolated. In embodiments, an autologous cancer cell vaccine may comprise additional components in addition to the tumor cells, such as a buffer and/or antisense nucleic acids. In embodiments, “autologous cancer cell vaccine” may refer to a biodiffusion chamber containing the tumor cells and one or more additional components. In certain aspects, the “autologous cancer cell vaccine” may be a “fully formulated chamber” also referred to herein as “fully formulated biodiffusion chamber.”

As used herein, the term “fully formulated chamber” or “fully formulated biodiffusion chamber” is a biodiffusion chamber that includes autologous tumor cells and other cells included in the tumor microenvironment (TME) that may or may not be treated prior to encapsulation in the chamber with a first amount of an IGF-1R AS ODN. The cells are encapsulated with exogenous addition of a second amount, for example at least 2 μg, at least 4 μg, at least 6 μg, at least 8 μg, or at least 10 μg, of IGF-1R AS ODN and the chamber is then irradiated with 5 Gy of gamma-irradiation.

As used herein, the term “small molecules” includes nucleic acids, peptides, proteins, and other chemicals (such as, for example, cytokines and growth hormones produced by cells), but does not include cells, exosomes, or microvesicles.

The term “targeting IGF-1R expression” or “targets IGF-1R expression” as used herein refers to administering an antisense nucleic acid that has a sequence designed to bind to the IGF-1R.

As used herein, the term “systemic administration” refers to achieving delivery of a substance throughout the body of a subject. Typical systemic routes of administration include parenteral administration, transdermal administration, intraperitoneal administration, intravenous administration, subcutaneous administration, and intramuscular administration.

Other administration routes include oral administration, nasal administration topical administration, intraocular administration, buccal administration, sublingual administration, vaginal administration, intraheptic, intracardiac, intrapancreatic, by inhalation, and via an implanted pump.

### Bladder Cancer

Bladder cancer is a type of cancer that is found in the lining of the urinary tract including the pelvis, ureters, bladder, and parts of the urethra. The most common form of bladder cancer is urothelial carcinoma. Bladder cancer occurs in people of all races and can affect people of any age. Bladder cancer or urothelial carcinoma is often described based on how far they have invaded the wall of the bladder. Papillary carcinomas, or non-invasive bladder cancer, grow in slender, finger-like projections from the inner surface of the bladder toward the hollow center. Papillary tumors often grow toward the center of the bladder without growing into the deeper bladder layers. Low-grade (slow growing), non-invasive papillary cancer tends to result in a more favorable prognosis. Flat carcinomas are another example of non-invasive bladder cancer. Flat carcinomas do not grow toward the hollow part of the bladder. If either a papillary or flat tumor grows into deeper layers of the bladder, it is called an invasive urothelial carcinoma. Invasive bladder cancers are more likely to spread and are much harder to treat.

### Antisense Molecules

Antisense molecules are nucleic acids that work by binding to a targeted complimentary sequence of mRNA by Watson and Crick base-pairing rules. The translation of target mRNA is inhibited by an active and/or passive mechanism when hybridization occurs between the complementary helices. In the passive mechanism, hybridization between the mRNA and exogenous nucleotide sequence leads to duplex formation that prevents the ribosomal complex from reading the message. In the active mechanism, hybridization promotes the binding of RnaseH, which destroys the RNA but leaves the antisense intact to hybridize with another complementary mRNA target. Either or both mechanisms inhibit translation of a protein contributing to or sustaining a malignant phenotype. As therapeutic agents, antisense molecules are far more selective and as a result, more effective and less toxic than conventional drugs.

The methods and compositions disclosed herein involve the use of antisense molecules for treating cancer. Typically, the antisense molecule is an antisense oligodeoxynucleotide (AS-ODN). In some embodiments, the antisense molecule comprises a modified phosphate backbone. In certain aspects, the phosphate backbone modification renders the antisense more resistant to nuclease degradation. In certain embodiments, the modification is a locked antisense. In other embodiments, the modification is a phosphorothioate linkage. In certain aspects, the antisense contains one or more phosphorothioate linkages. In certain embodiments, the phosphorothioate linkages stabilize the antisense molecule by conferring nuclease resistance, thereby increasing its half-life. In some embodiments, the antisense may be partially phosphorothioate-linked. For example, up to about 1%, up to about 3%, up to about 5%, up to about 10%, up to about 20%, up to about 30%, up to about 40%, up to about 50% up to about 60%, up to about 70%, up to about 80%, up to about 90%, up to about 95%, or up to about 99% of the antisense may be phosphorothioate-linked. In some embodiments, the antisense is fully phosphorothioate-linked. In other embodiments, phosphorothioate linkages may alternate with phosphodiester linkages. In certain embodiments, the antisense has at least one terminal phosphorothioate monophosphate.

In some embodiments, the antisense molecule comprises one or more CpG motifs. In other embodiments, the antisense molecule does not comprise a CpG motif. In certain aspects, the one or more CpG motifs are methylated. In other aspects, the one or more CpG motifs are unmethylated. In certain embodiments, the one or more unmethylated CpG motifs elicit an innate immune response when the antisense molecule is administered to a subject. In some aspects, the innate immune response is mediated by binding of the unmethylated CpG-containing antisense molecule to Toll like Receptors (TLR).

In certain embodiments, the antisense molecule comprises at least one terminal modification or “cap”. The cap may be a 5′ and/or a 3′-cap structure. The terms “cap” or “end-cap” include chemical modifications at either terminus of the oligonucleotide (with respect to terminal ribonucleotides), and including modifications at the linkage between the last two nucleotides on the 5′ end and the last two nucleotides on the 3′ end. The cap structure may increase resistance of the antisense molecule to exonucleases without compromising molecular interactions with the target sequence or cellular machinery. Such modifications may be selected on the basis of their increased potency in vitro or in vivo. The cap can be present at the 5′-terminus (5′-cap) or at the 3′-terminus (3′-cap) or can be present on both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety. The phosphorothioate or phosphorodithioate linkage(s), when part of a cap structure, are generally positioned between the two terminal nucleotides on the 5′ end and the two terminal nucleotides on the 3′ end.

In preferred embodiments, the antisense molecule targets the expression of Insulin like Growth Factor 1 Receptor (IGF-1R). IGF-1R is a tyrosine kinase cell surface receptor that shares 70% homology with the insulin receptor. When activated by its ligands (IGF-I, IGF-II and insulin), it regulates broad cellular functions including proliferation, transformation and cell survival. The IGF-1R is not an absolute requirement for normal growth, but it is essential for growth in anchorage-independent conditions that may occur in malignant tissues. A review of the role of IGF-1R in tumors is provided in Baserga et al., Vitamins and Hormones, 53:65-98 (1997), which is incorporated herein by reference in its entirety.

In certain embodiments, the antisense molecule is an oligonucleotide directed against DNA or RNA of a growth factor or growth factor receptor, such as, for example, IGF-1R.

In certain embodiments, the antisense is a deoxynucleotide directed against IGF-1R (IGF-1R AS ODN). The full length coding sequence of IGF-1R is provided as SEQ ID NO:15 (see, for example, PCT/US2016/26970, which is incorporated herein by reference in its entirety).

In certain embodiments, the antisense molecule comprises nucleotide sequences complementary to the IGF-1R signal sequence, comprising either RNA or DNA. The signal sequence of IGF-1R is a 30 amino acid sequence. In other embodiments, the antisense molecule comprises nucleotide sequences complementary to portions of the IGF-1R signal sequence, comprising either RNA or DNA. In some embodiments, the antisense molecule comprises nucleotide sequences complementary to codons 1-309 of IGF-1R, comprising either RNA or DNA. In other embodiments, the antisense molecule comprises nucleotide sequences complementary to portions of codons 1-309 of IGF-1R, comprising either RNA or DNA.

In certain embodiments, the IGF-1R AS ODN is at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, or at least about 50 nucleotides in length. In some embodiments, the IGF-1R AS ODN is from about 15 nucleotides to about 22 nucleotides in length. In certain aspects, the IGF-1R AS ODN is about 18 nucleotides in length.

In certain embodiments, the IGF-1R AS ODN forms a secondary structure at 18 °C, but does not form a secondary structure at about 37 °C. In other embodiments, the IGF-1R AS ODN does not form a secondary structure at about 18 °C or at about 37 °C. In yet other embodiments, the IGF-1R AS ODN does not form a secondary structure at any temperature. In other embodiments, the IGF-1R AS ODN does not form a secondary structure at 37 °C. In particular embodiments, the secondary structure is a hairpin loop structure.

In some aspects, the IGF-1R AS ODN comprises the nucleotide sequence of SEQ ID NO:1, or a fragment thereof. In certain embodiments, the IGF-1R AS ODN may have at least about 70%, at least about 75%, at least about 80%, at least about 85%, at least about 90%, at least about 95%, at least about 96%, at least about 98%, or 100% identity to SEQ ID NO: 1, or a fragment thereof. In some embodiments, the IGF-1R AS ODN comprises one or more phosphorothioate linkages (see, e.g., SEQ ID NO: 16).

In certain aspects, the IGF-1R AS ODN consists of SEQ ID NO: 1. NOBEL is an 18-mer oligodeoxynucleotide with a phosphorothioate backbone and a sequence complimentary to codons 2 through 7 in the IGF-1R gene. As such, NOBEL is an antisense oligonucleotide directed against IGF-1R (IGF-1R AS ODN). The NOBEL sequence, derived as the complimentary sequence of the IGF-1R gene at the 5′ end, is:

(SEQ ID NO: 1)
5′-TCCTCCGGAGCCAGACTT-3′.

NOBEL has a stable shelf life and is resistant to nuclease degradation due to its phosphorothioate backbone. Administration of NOBEL can be provided in any of the standard methods associated with introduction of oligodeoxynucleotides known to one of ordinary skill in the art. Advantageously, the AS ODNs disclosed herein, including NOBEL, may be administered with little/no toxicity. Even levels of about 2 g/kg (scaled) based on mice tests (40 μg in the tail vain) did not reveal toxicity issues. NOBEL can be manufactured according to ordinary procedures known to one of ordinary skill in the art.

The antisense molecule, for example the NOBEL sequence of SEQ ID NO: 1, may also comprise one or more p-ethoxy backbone modifications as disclosed in U.S. Pat. No. 9,744,187, which is incorporated by reference herein in its entirety. In some embodiments, the nucleic acid backbone of the antisense molecule comprises at least one p-ethoxy backbone linkage. For example, up to about 1%, up to about 3%, up to about 5%, up to about 10%, up to about 20%, up to about 30%, up to about 40%, up to about 50%, up to about 60%, up to about 70%, up to about 80%, up to about 90%, up to about 95%, or up to about 99% of the antisense molecule may be p-ethoxy-linked. The remainder of the linkages may be phosphodiester linkages or phosphorothioate linkages or a combination thereof. In a preferred embodiment 50% to 80% of the phosphate backbone linkages in each oligonucleotide are p-ethoxy backbone linkages, wherein 20% to 50% of the phosphate backbone linkages in each oligonucleotide are phosphodiester backbone linkages.

Various IGF-1R antisense sequences are bioactive in some or all of the multi-modality effects of the NOBEL sequence. The 18-mer NOBEL sequence has both IGF-1R receptor downregulation activity as well as TLR agonist activity, and further experimentation in mice suggests that both activities are necessary for in vivo anti-tumor immune activity. While the AS ODN molecule has anti-tumor activity, the complimentary sense sequence does not, despite also having a CpG motif.

In certain embodiments, the sequence of the antisense is selected from the group consisting of SEQ ID NOS 1-14, as shown in Table 1. In some embodiments, the antisense has 90% sequence identity to one or more of SEQ ID NOS 1-14. In some embodiments, the antisense has 80% sequence identity to one or more of SEQ ID NOS 1-14. In some embodiments, the antisense has 70% sequence identity to one or more of SEQ ID NOS 1-14.

TABLE 1
Additional downstream sequences for
IGF-IR AS ODN Formulation

| | Corresponds | SEQ |
|---|---|---|
| | to IGF-1R | ID |
| Sequences with ACGA Motif | Codons | NO: |
| 5′-TCCTCCGGAGCCAGACTT-3′ | 2-7 | 1 |
| 5′-TTCTCCACTCGTCGGCC-3′ | 26-32 | 2 |
| 5′-ACAGGCCGTGTCGTTGTC-3′ | 242-248 | 3 |
| 5′-GCACTCGCCGTCGTGGAT-3′ | 297-303 | 4 |
| 5′-CGGATATGGTCGTTCTCC-3′ | 589-595 | 5 |
| 5′- TCTCAGCCTCGTGGTTGC-3′ | 806-812 | 6 |
| 5′-TTGCGGCCTCGTTCACTG-3′ | 1,033-1,039 | 7 |
| 5′-AAGCTTCGTTGAGAAACT-3′ | 1,042-1,048 | 8 |
| 5′-GGACTTGCTCGTTGGACA-3′ | 1,215-1,221 | 9 |
| 5′-GGCTGTCTCTCGTCGAAG-3′ | 1,339-1,345 | 10 |
| 5′-CAGATTTCTCCACTCGTCGG-3′ | 27-34 | 11 |
| 5′-CCGGAGCCAGACTTCAT-3′ | 1-6 | 12 |
| 5′-CTGCTCCTCCTCTAGGATGA-3′ | 407-413 | 13 |
| 5′-CCCTCCTCCGGAGCC-3′ | 4-8 | 14 |

In certain embodiments, the IGF-1R AS ODN comprises the nucleotide sequence of any one of SEQ ID NOs: 1-14, or fragments thereof. In certain embodiments, the IGF-1R AS ODN may have at least about 70%, at least about 75%, at least about 80%, at least about 85%, at least about 90%, at least about 95%, at least about 96%, at least about 98%, or 100% identity to any one of SEQ ID NOs: 1-14, or fragments thereof.

In some embodiments, the antisense molecule downregulates the expression of genes downstream of IGF-1R pathway in a cell. In certain aspects, the downstream gene is hexokinase (Hex II). In some embodiments, the antisense molecule downregulates the expression of housekeeping genes in the cell. In some aspects, the housekeeping gene is L13.

In certain aspects, the IGF-1R AS ODN is chemically synthesized. In certain embodiments, the IGF-1R AS ODN is manufactured by solid phase organic synthesis. In some aspects, the synthesis of the IGF-1R AS ODN is carried out in a synthesizer equipped with a closed chemical column reactor using flow-through technology. In some embodiments, each synthesis cycle sequence on the solid support consists of multiple steps, which are carried out sequentially until the full-length IGF-1R AS ODN is obtained. In certain embodiments, the IGF-1R AS ODN is stored in a liquid form. In other embodiments, the IGF-1R AS ODN is lyophilized prior to storing. In some embodiments, the lyophilized IGF-1R AS ODN is dissolved in water prior to use. In other embodiments, the lyophilized IGF-1R AS ODN is dissolved in an organic solvent prior to use. In yet other embodiment, the lyophilized IGF-1R AS ODN is formulated into a pharmaceutical composition. In some aspects the pharmaceutical composition is a liquid pharmaceutical composition. In other aspects, the pharmaceutical composition is a solid pharmaceutical composition. Additional antisense nucleic acids are also described in U.S. Publication No. 2017/0056430, which is incorporated herein by reference in its entirety.

### Autologous Cancer Cell Vaccine

### Introduction

Immunotherapy is currently used to target hematologic malignancies with one common cellular antigen. Unfortunately, solid tumors are far more complex, representing epigenetic progression of genetic changes to a malignant state with an unidentifiable number of tumor-specific targets. Even more challenging, within a WHO diagnostic cancer group there exists marked variations in tumor phenotypes. An autologous cell vaccine would encompass all such variations and all such targets and represent an ideal subject-specific immunotherapy for solid tumor cancers. An autologous cancer cell vaccine however, cannot be derived from primary cell cultures because serial passages alter the tumor phenotype thus diminishing the array of tumor-specific antigens. This would also require impossible lot-release qualification at each passage. The present disclosure eliminates these concerns by plating freshly resected, morselized tumor cells and reimplanting them within 24 hours as a depot antigen. In certain aspects, the excellent results achieved herein are obtained by ensuring that an appropriate number of cells are present in the chamber(s), among other specifics described herein.

Previous studies have designed autologous cell vaccine through the use of antigen presenting cells, instead of autologous tumor cells. In this paradigm, a subject's monocytes are collected from a pre-treatment plasma leukopheresis and differentiated into autologous dendritic cells (DC) ex vivo. The dendritic cells are then presented with the subject's tumor crude lysate inducing DC activation/maturation, and at a later time point, the matured dendritic cells, now cross-primed with tumor antigens are injected in the subject as a DC vaccine. Ex vivo differentiation, however, is missing a number of key stimulatory components only occurring in vivo. In addition, differentiation of DCs from hematopoietic precursors requires extensive in vitro manipulations with labor-intensive cell processing in expensive facilities. The present disclosure obviates these concerns by providing an endogenous DC maturation process and an immunomodulatory and immunostimulatory antisense oligodeoxynucleotide (AS-ODN) that promotes the development of an appropriate immune response. More specifically, the present disclosure provides a biodiffusion chamber comprising dispersed tumor cells derived from the patient and irradiated antisense molecules, which is implanted into the patient for therapeutically effective time. Without being bound by any theory, it is thought that the combination of irradiated tumor cells, antisense, and biodiffusion chamber act in concert to simulate the local immune response, and enhance the response by reducing or eliminating M2 cells, preventing dampening of the immune system.

Thus, the present disclosure shows that an irradiated, implantable biodiffusion chamber comprising freshly resected tumor cells and IGF-1R AS ODN safely serves as an effective, subject-specific autologous cell vaccine for bladder cancer immunotherapy. As such, the use of the claimed implantable biodiffusion chamber to mount an immune response that selectively targets tumor cells in a subject provides a new and significant approach for the treatment of bladder cancer, including metastatic bladder cancer.

### Biodiffusion Chamber

A representative diffusion chamber comprises a chamber barrel having two ends, a first end and a second end. In embodiments, the biodiffusion chamber is a small ring capped on either side by a porous, cell-impermeable membrane, such as the Duropore membrane manufactured by Millipore Corporation. Optionally, one of the ends may be closed off as part of the chamber body leaving only one end open to be sealed using the porous membrane. The membranes can be made of plastic, teflon, polyester, or any inert material which is strong, flexible and able to withstand chemical treatments. The chamber can be made of any substance, such as and not limited to plastic, teflon, lucite, titanium, Plexiglass or any inert material which is non-toxic to and well tolerated by humans. In addition, the chambers should be able to survive sterilization. In some aspects, the diffusion chambers are sterilized with ethylene oxide prior to use. Other suitable chambers are described in U.S. Prov. No. 62/621,295, filed Jan. 24, 2018, U.S. Pat. No. 6,541,036, PCT/US16/26970, and U.S. Pat. No. 5,714,170, which are each incorporated herein by reference in their entirety.

In certain embodiments, the membrane allows passage of small molecules but does not allow passage of cells (i.e., the cells cannot leave or enter the chamber). In some aspects, the diameter of the pores of the membrane allows nucleic acids and other chemicals (such as, for example, cytokines produced by cells) to diffuse out of the chamber, does not allow passage of cells between the chamber and the subject in which it is implanted. The biodiffusion chambers useful in the present disclosure include any chamber which does not allow passage of cells between the chamber and the subject in which it is implanted, provided however, that the chamber permits interchange and passage of factors between the chamber and the subject. Thus, in certain aspects, the pore size has a cut-off that prevent passage of materials that are greater than 100 μm³ in volume into and out of the chamber. In some embodiments, the pores of the membrane have a diameter of about 0.25 μm or smaller. For example, the pores may have a diameter of about 0.1 μm. In particular aspects, the pores range in diameter from 0.1 μm to 0.25 μm. See also, Lange, et al., J. Immunol., 1994, 153, 205-211 and Lanza, et al., Transplantation, 1994, 57, 1371-1375, each of which is incorporated herein by reference in their entireties. This pore diameter prevents the passage of cells in or out of the chamber. In certain embodiments, diffusion chambers are constructed from 14 mm Lucite rings with 0.1 μm pore-sized hydrophilic Durapore membranes (Millipore, Bedford, Mass.).

In certain embodiments, a biodiffusion chamber comprises a membrane that allows the IGF-1R AS ODN to diffuse out of the chamber. In some embodiments, about 50% of the IGF-1R AS ODN diffuses out of the chamber in about 12 hours, about 60% of the IGF-1R AS ODN diffuses out of the chamber in about 24 hours, about 80% of the IGF-1R AS ODN diffuses out of the chamber in about 48 hours, and/or about 100% of the IGF-1R AS ODN diffuses out of the chamber in about 50 hours.

In an exemplary approach, to assemble the biodiffusion chamber, a first porous membrane is attached to one side of a first diffusion chamber, using glue and pressure to create a tight seal. A second porous membrane is similarly attached to a second diffusion chamber ring. The membranes can be secured in position with rubber gaskets which may also provide a tighter seal. The diffusion chamber rings are left overnight (minimum 8 hours) to dry. Then, the first diffusion chamber ring and the second diffusion chamber ring are attached to one another using glue and left overnight (minimum 8 hours) to dry. In a preferred embodiment, the first chamber ring and second chamber ring joining process comprises using 2 dichloroethane as a solvent to facilitate adhesion between the two rings. In an alternative approach, the chamber may have only one side that contains a porous membrane.

On the barrel portion of the chamber, one or more openings (e.g. ports) are provided which can be covered by a cap which is accessed from outside of the subject's body once the chamber is implanted, thus allowing the diffusion chamber to be refilled. The openings allow for multiple and sequential sampling of the contents, without contamination and without harming the subject, therefore significantly reducing the number of implantation procedures performed on the subject. Before implantation into the patient, the one or more openings may be sealed with bone wax, a port plug or cap made from, for example, PMMA. The cap can be a screw-on type of self-sealing rubber and fitted to the opening. In some configurations, the diffusion chamber may contain two or more injection openings or ports. Sampling of the chamber contents can be performed by accessing the opening by removing the cap on the outside of the subject's body and inserting an ordinary needle and syringe. In some embodiments, the chamber may further include a removal device. Such a device facilitates removal of the chamber from the patient.

In embodiments, the chamber serves as an antigen depot designed so that tumor antigens diffuse out of the chamber for the purpose of promoting a therapeutic host immune response. Exogenous IGF-1R AS ODN and ex vivo irradiation promote a pro-inflammatory response. This formulation is associated with clinical and radiographic improvements, prolonged survival on protocol, and represents a novel autologous cell vaccine that includes an exogenous active pharmaceutical ingredient (API) and radiation that we interpret as inducing or enhancing tumor immunity effect. Furthermore the addition of low concentration of the IGF-1R AS ODN is critical to a pro-inflammatory response.

In certain embodiments the disclosure provides a biodiffusion chamber for implantation into a subject suffering from cancer comprising: (a) tumor cells; and (b) an effective amount of an antisense molecule. In other embodiments is provided a method for treating cancer in a subject comprising: (a) obtaining a biodiffusion chamber comprising tumor cells and an effective amount of an antisense nucleic acid; (b) irradiating the biodiffusion chamber and contents; and (c) implanting the irradiated biodiffusion chamber into the subject for a therapeutically effective time.

In certain embodiments, the IGF-1R AS ODN is present in the biodiffusion chamber in an amount ranging from about 0.5 μg to about 10 μg. In certain aspects, the IGF-1R AS ODN is present in an amount ranging from about 1 μg to about 5 μg per chamber, or from about 2 μg to about 4 μg per chamber. For example, the IGF-1R may be present in an amount of about 1, about 1.5, about 2, about 2.5, about 3, about 3.5, about 4, about 4.5, or about 5 μg per chamber. In specific aspects, the IGF-1R AS ODN is present in an amount of about 2 μg per chamber. In specific aspects, the IGF-1R AS ODN is present in an amount of about 4 μg per chamber. In certain embodiments, the IGF-1R AS ODN is present in the biodiffusion chamber in an amount ranging from about 10 μg to about 500 μg. In certain aspects, the IGF-1R AS ODN is present in an amount ranging from about 40 μg to about 400 μg per chamber, from about 40 to about 70 μg per chamber, or from about 100 μg to about 200 μg per chamber. In specific aspects, the IGF-1R AS ODN is present in an amount of about 100 μg per chamber. In specific aspects, the IGF-1R AS ODN is present in an amount of about 200 μg per chamber. In certain aspects, the IGF-1R AS ODN is present in an amount ranging from about 1 mg to about 100 mg per chamber, or from about 2 mg to about 10 mg per chamber. In specific aspects, the IGF-1R AS ODN is present in an amount of about 2 mg per chamber. In specific aspects, the IGF-1R AS ODN is present in an amount of about 4 mg per chamber. Without being bound by any theory it is thought that these levels promote an enhanced Th1 response in a subject, while avoiding an M2 immunostimulatory response in the subject.

In certain embodiments, the tumor cells are not treated with an IGF-1R AS ODN prior to encapsulation in the chamber. Typically, however, the tumor cells are treated with an IGF-1R AS ODN prior to encapsulation in the chamber. The time for treating the cells pre-encapsulation may vary. For example, the tumor cells may be treated ex vivo with an IGF-1R AS ODN immediately before encapsulation, for up to about 4 hours, for up to about 6 hours, for up to about 8 hours, for up to about 12 hours or for up to about 18 hours. Typically, the tumor tissue may be treated ex vivo for about 12 hours to about 18 hours pre-encapsulation. Conveniently, the cells may be encapsulated after a pre-treatment lasting up to overnight. Without being bound by any theory, it is thought that the pre-encapsulation treatment plays a desirable role in stimulating production of tumor antigen.

The amount of IGF-1R AS ODN used for the pre-encapsulation treatment may be in a range of about 1 mg to 8 mg per million cells; for example, about 2 mg to about 6 mg per million cells, about 3 mg to about 5 mg per million cells. Typically the amount of IGF-1R AS ODN used for treatment prior to encapsulation is about 4 mg per million cells.

In some embodiments, the IGF-1R AS ODN for ex vivo treatment of the tumor cells is used at a concentration ranging from about at least 2 mg/ml to at least about 5 mg/ml. In certain aspects, the IGF-1R AS ODN is used at a concentration of at least 4 mg/ml. In specific embodiments, the IGF-1R AS ODN is used at a concentration of 4 mg/ml.

In certain embodiments, the IGF-1R AS ODN used to treat tumor cells ex vivo and the IGF-1R AS ODN present in the chamber are the same. In other embodiments, the IGF-1R AS ODN used to treat tumor cells ex vivo and the IGF-1R AS ODN present in the chamber are different. In certain embodiments, the IGF-1R AS ODN used to treat tumor cells ex vivo is at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, or at least about 50 nucleotides in length. In some embodiments, the IGF-1R AS ODN used to treat tumor cells ex vivo is from about 15 nucleotides to about 22 nucleotides in length. In certain aspects, the IGF-1R AS ODN used to treat tumor cells is about 18 nucleotides in length.

In certain embodiments, the IGF-1R AS ODN used to treat tumor cells ex vivo forms a secondary structure at 18 °C, but does not form a secondary structure at about 37 °C. In other embodiments, the IGF-1R AS ODN used to treat tumor cells does not form a secondary structure at about 18 °C or at about 37 °C. In yet other embodiments, the IGF-1R AS ODN used to treat tumor cells ex vivo does not form a secondary structure at any temperature. In other embodiments, the IGF-1R AS ODN used to treat tumor cells does not form a secondary structure at 37 °C. In particular embodiments, the secondary structure is a hairpin loop structure.

In some aspects, the IGF-1R AS ODN used to treat tumor cells comprises the nucleotide sequence of SEQ ID NO:1, or a fragment thereof. In certain embodiments, the IGF-1R AS ODN used to treat tumor cells may have at least about 70%, at least about 75%, at least about 80%, at least about 85%, at least about 90%, at least about 95%, at least about 96%, at least about 98%, or 100% identity to SEQ ID NO: 1, or a fragment thereof. In certain aspects, the IGF-1R AS ODN used to treat tumor cells is SEQ ID NO: 1.

After the tumor cells are treated with the AS-ODN for a period of time, the AS-ODN is removed and fresh AS-ODN is added to the chamber, which is then irradiated prior to implantation into a subject.

In certain aspects, the biodiffusion chamber is treated with gamma irradiation at an amount of about 1 Gy, about 2 Gy, about 4 Gy, about 5 Gy, about 6 Gy, about 10 Gy, or up to about 15 Gy. In certain aspects, the dose of radiation is not more than about 5 Gy. In other aspects, the dose of radiation is at least about 5 Gy. In some aspects, the dose of radiation is 5 Gy. In certain embodiments, the biodiffusion chamber may be irradiated at least once, at least twice, at least three times, at least four times, or at least five times. In some embodiments, the chamber is irradiated less than about 24 hours prior to implantation into a subject. In other embodiments, chamber is irradiated about 24 hours prior to implantation into the subject. In yet other embodiments, the chamber is irradiated at least about 24 hours prior to implantation into the subject. In still other embodiments, the chamber is irradiated not more than about 48 hours prior to implantation into the subject. In yet other embodiments, the chamber is irradiated at least about 48 hours prior to implantation into the subject.

While the tumor cells are typically killed prior to implantation; for example by radiation, the cells need not be killed and indeed it may be advantageous to maintain the cells in an alive state to promote release of antigen. Thus, in certain embodiments, the cells may not be irradiated prior to implantation. For safety purposes, however, it is desirable to prevent release of live tumor cells into the subject.

Tumor cells can be placed in a diffusion chamber in varying numbers. In certain embodiments, about 1×10⁴ to about 5×10⁶ tumor cells are placed in each diffusion chamber. In other embodiments, about 1×10⁵ to about 1.5×10⁶ tumor cells are placed in the diffusion chamber. In yet other embodiments, about 5×10⁵ to 1×10⁶ tumor cells are placed in the chamber. with a subject can be used. We have discovered that the number of tumor cells can impact the subjects' anti-tumor response and that an appropriate range should be selected to increase the chance to obtain the desired results. In some embodiments, the anti-tumor immune response is optimal in a range of about 750,000 to about 1,250,000 cells in a chamber, with a peak at about 1 million cells/chamber. Multiple chambers containing irradiated tumor cells are administered and to maintain the optimal immune the response the number of cells/chamber is preferably maintained within the range. Preferably, the tumor cells are intact and not autolyzed or otherwise damaged as described herein.

In certain embodiments, it may be preferable to maintain the ratio of cells to AS ODN in a chamber. Thus, in certain aspects a chambers may contain about 2 μg of AS ODN and between 750,000 and 1,250,000 cells; for example 1,000,000 cells. The ratio of cells to AS ODN may thus be in a range from about 3.75×10⁵ to about 6.25×10⁵ per μg AS ODN; for example, about 5.0×10⁵ cells per μg. Thus, in a typical patient receiving 20 chambers the total dose of AS ODN is about 40 μg.

Typically, administration will be in a chamber as described herein; however, in certain aspects, the irradiated cells and IGF-1R AS ODN may be co-administered to the subject without being contained physically together in the chamber or another container. In certain methods using this approach, the irradiated cells IGF-1R AS ODN thus disperse, diffuse, or are metabolized in the body limited by the physiology of the subject. Thus, in certain aspects, e.g. the tumors cells for use may be prepared as described herein for the chamber and administered with the IGF-1R AS ODN but the administration may be not contained within a physical container. Such administration is typically intramuscular.

### Tumor Tissue Preparation for Chamber

Tumor cells for use in the autologous vaccination are surgically removed from the subject. In embodiments, the tumor cells are removed from the patient using a tissue morselator. The extraction device preferably combines a high-speed reciprocating inner cannula within a stationary outer cannula and electronically controlled variable suction. The outer cannula has a diameter of 1.1 mm, 1.9 mm, 2.5 mm, or 3.0 mm, and a length of 10 cm, 13 cm, or 25 cm. The instrument also relies on a side-mouth cutting and aspiration aperture located 0.6 mm from the blunt desiccator end. The combination of gentle forward pressure of the aperture into the tissue to be removed and suction draws the desired tissue into the side aperture, allowing for controlled and precise tissue resection through the reciprocal cutting action of the inner cannula. A key feature is the absence of a rotation blade; this avoids drawing unintended tissue into the aperture. An example of a suitable device is the Myriad® tissue aspirator (NICO Corporation® Indianapolis, IN), a minimally invasive surgical system which may be used for the removal of soft tissues with direct, microscopic, or endoscopic visualization. The shaved tissue is suctioned, gathered in to a collection chamber, and is collected in a sterile tissue trap. During collection of the tissue in the sterile tissue trap, blood is removed from the preparation. Preferably, the sterile trap contains a collection dish at the bottom of the trap and a stem that provides access to the trap. The trap structure may also contain an inner ladle-shaped structure that is removable from the trap to facilitate tissue removal from the trap.

Preferably, the morselator generates no heat at the resection site or along its shaft, and requires no ultrasonic energy for tissue removal. Thus, in particular embodiments, the tumor tissue is morselized tumor tissue (i.e. tumor shaved tissue obtained by side-mouth cutting in the absence of heat, and optionally in the absence of ultrasonic treatment). Advantageously, the aspirator-extract and morselized tissue has higher viability than tissue removed by other methods. It is believed that the extraction process maintains higher tumor cell viability in part due to restricting exposure of the tumor cells to high temperatures during removal. For example, the methods herein do not expose tumor cells to above 25 °C during removal. Thus, the cells are not exposed to temperatures above body temperature, i.e., about 37 °C.

In some embodiments, the tumor cells are viable when they are removed from the patient, for example, using a tissue morcellator. In some embodiments, removal of the cells from the patient using a tissue morcellator does not kill the cells. In some embodiments, at least 60%, at least 60%, at least 70%, at least 80%, at least 90%, at least 95%, at least 98, or at least 99% of the tumor cells removed from the patient using a tissue morcellator are viable.

The amount of tumor tissue obtained from the subject may vary. Preferably, the amount is at least 1, at least 2, at least 3 grams or at least 4 grams of wet tumor tissue is obtained from the patient. The tissue is removed from the sterile tissue trap and disaggregated by pipetting with a sterile pipette to break up large tissue fragments. The disaggregated cell suspension is then placed onto sterile tissue culture plates in serum-containing media, and incubated in a tissue culture incubator. This plating step serves to enrich the desired functional cells by adherence, and also helps to remove debris from the preparation. Thus, the tumor cells used in treatments described herein preferably consist essentially of, or consist of, adherent cells from the tumor tissue.

After a predetermined incubation time (e.g., 6, 12, 24, or 48 hours), the cells are removed from the plates. The cells may be removed by scraping, by chemical methods (e.g. EDTA) or by enzymatic treatment (e.g. trypsin). The cells are placed into one or more diffusion chambers. In some embodiments, the cells are split between 2, 3, 4, 5, 6, 7, 8, 9, 10, 11, 12, 13, 14, 15, 16, 17, 18, 19, 20, 21, 22, 23, 24, 25, 26, 27, 28, 29, 30 or more diffusion chambers. Often, 20 chambers are used. In some embodiments, each diffusion chamber contains an equal number of cells. In some embodiments, a first diffusion chamber contains more cells than a second chamber.

In some embodiments, the cells are sorted before being placed in the chamber. In some embodiments, the cells are enriched by selecting for one or more cellular markers before being placed in the chamber. The selection may be performed, for example, using beads or by cell sorting techniques known to those of skill in the art. In some embodiments, the cells placed into the chamber are enriched for one or more markers.

In some embodiments, implantation of the biodiffusion chamber for a therapeutically effective time reduces or eliminates return of the cancer in the subject. In certain aspects, implantation of the biodiffusion chamber causes a reduction of tumor volume associated with the cancer in the subject. In yet other embodiments, implantation of the biodiffusion chamber for a therapeutically effective time induces elimination of the tumor in the subject. In some embodiments, implantation of the chamber inhibits regrowth of the tumor for at least 3 months, at least 6 months, at least 12 months, at least 36 months, or indefinitely.

The biodiffusion chamber can be implanted in a subject in the following non-limiting ways: subcutaneously, intraperitoneally, and intracranially. In certain embodiments, the diffusion chamber(s) is implanted into an acceptor site of the body having good lymphatic drainage and/or vascular supply such as the rectus sheath. In other embodiments, a refillable chamber can be employed such that the diffusion chamber can be re-used for treatments and emptied following treatments. In certain aspects, a plurality of diffusion chambers, preferably between 5 and 20, can be used in a single subject.

In certain embodiments, at least about 1, at least about 2, at least about 3, at least about 4, at least about 5, at least about 10, at least about 15, at least about 20, at least about 25, at least about 30, at least about 35, at least about 40, at least about 45, or at least about 50 chambers are implanted into the subject. In some embodiments, 10-20 chambers are implanted into the subject. Preferably, about 20 chambers are implanted into the subject. In certain embodiments, the tumor cells are divided equally among each chamber.

Typically, the chamber is removed after period of time. For example, the chamber may be implanted in the subject for about 24 hours, about 48 hours, about 72 hours, or about 96 hours. Implantation for about 48 hours is associated with beneficial therapeutic outcomes. Accordingly, the preferred time of implantation is about 48 hours. In certain embodiments, the vaccination procedure is performed one time per patient. In other embodiments, the vaccination procedure is performed multiple times per patient. In embodiments, the vaccination procedure is performed two times, three times, four times, five times, six times, seven times, or eight times in a single patient. In embodiments, the vaccination is repeated every 7, 14, or 28 days, or every 1, 3, or 6 months for a given period of time. In further embodiments, the vaccination procedure is repeated periodically until the patient is free of cancer.

Without being bound by any theory, it is thought that implantation of the biodiffusion chamber causes elimination or reduction of M2 cells at or near the implantation site such that an immune response against tumor antigens diffusing out from the chamber is achieved. In certain aspects, elimination or reduction of M2 cells at the implantation site leads to enhanced presentation of autologous tumor antigens by antigen-presenting cells (APC) to CD4 T cells leading to production of interferon-gamma (IFNγ) and the induction of type 1 tumor immunity. In certain aspects, the production of IFNγ by tumor antigen-specific CD4 T cells and the anti-M2 effects of IGF-1R AS ODN drive type 1 anti-tumor immunity and the loss of anti-inflammatory M2 cells from the circulation and tumor microenvironment indirectly interfering with tumor growth. In some aspects, the production of IFNγ by tumor antigen-specific CD4 T cells and the anti-M2 effects of IGF-1R AS ODN unleashes effector-mediated damage to the tumor cells and tumor microenvironment (M2 cells) and initiates the longer process of programming memory T cells recognizing tumor antigens. In certain embodiments, the anti-tumor adaptive immune response sustains continued tumor regression.

Optionally, the cells introduced into the chamber may be enriched for certain cell types. Nestin a, cytoskeleton-associated class VI intermediate filament (IF) protein, has traditionally been noted for its importance as a neural stem cell marker. We have discovered that in certain tumor samples, cells positive for nestin (nestin+ cells) are enriched compared to benign tissue, and that this associated corresponds to improved therapeutic response. Thus, in certain aspects, a subject's tumor can be biopsied to assess the degree of nestin expression, and therefore, in certain aspects, the chamber cells are enriched Nestin-positive (“+”) cells compared to benign tissue. Without being bound by any theory, it is thought that nestin provides a marker associated with antigens suitable useful in producing an anti-tumor immune response. Accordingly, the cells implanted into the chamber may be enriched for nestin+ cells compared to the tumor cell population as a whole when extracted from the subject.

### Compositions

In some embodiments, the present disclosure provides a composition comprising cancer cells (e.g., bladder cancer cells). In some embodiments, the present disclosure provides a composition comprising cancer cells (e.g., bladder cancer cells) and antisense (e.g., IGF-1R AS ODN). In some embodiments, the composition comprises morselized cancer cells (e.g., bladder cancer cells) and antisense (e.g., IGF-1R AS ODN). In some embodiments, the composition comprises cancer cells (e.g., bladder cancer cells) and antisense (e.g., IGF-1R AS ODN), wherein one or both of the cancer cells and antisense is irradiated. In some embodiments, the composition comprises irradiated cancer cells (e.g., bladder cancer cells) and irradiated antisense (e.g., IGF-1R AS ODN).

In some embodiments, the composition comprises about 1×10⁴, about 5×10⁴, about 1×10⁵, about 5×10⁵, about 1×10⁶, about 5×10⁶, about 1×10⁷, about 5×10⁷, about 1×10⁸, about 5×10⁸, about 1×10⁹, about 5×10⁹, about 1×10¹⁰, about 5×10¹⁰ cancer cells. In some embodiments, the composition comprises about 1 μg, about 2 μg, about 3 μg, about 4 μg, about 5 μg, about 6 μg, about 7 μg, about 8 μg, about 9 μg, about 10 μg, about 20 μg, about 30 μg, about 40 μg, about 50 μg, about 60 μg, about 70 μg, about 80 μg, about 90 μg, about 100 μg, about 250 μg, about 500 μg, about 750 μg, about 1 mg, about 2 mg, about 3 mg, about 4 mg, about 5 mg, about 6 mg, about 7 mg, about 8 mg, about 9 mg, or about 10 mg antisense (e.g., IGF-1R AS-ODN). In some embodiments, the composition comprises about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 250 μg/ml, about 500 μg/ml, about 750 μg/ml, or about 1 mg/mL antisense (e.g., IFG-1R AS-ODN).

In some embodiments, the compositions comprise cancer cells (e.g., bladder cancer cells) and antisense (e.g., IGF-1R AS ODN), and further comprise a pharmaceutically acceptable carrier, buffer, stabilizer, or excipient.

Also provided are biodiffusion chambers comprising the compositions of the disclosure.

### Systemic Administration

As an alternative to, or supplement to, implantation of the chambers, IGF-1R AS ODN may be administered systemically. Thus, in embodiments, the IGF-1R AS ODN is provided in a pharmaceutical composition for systemic administration. In addition to the IGF-1R AS ODN, the pharmaceutical composition may comprise, for example, saline (0.9% sodium chloride). The composition may comprise phospholipids. In some aspects, the phospholipids are uncharged or have a neutral charge at physiologic pH. In some aspects, the phospholipids are neutral phospholipids. In certain aspects, the neutral phospholipids are phosphatidylcholines. In certain aspects, the neutral phospholipids are dioleoylphosphatidyl choline (DOPC). In some aspects, the phospholipids are essentially free of cholesterol.

In some aspects, the phospholipids and oligonucleotides are present at a molar ratio of from about 5:1 to about 100:1, or any ratio derivable therein. In various aspects, the phospholipids and oligonucleotides are present at a molar ratio of about 5:1, about 10:1, about 15:1, about 20:1, about 25:1, about 30:1, about 35:1, about 40:1, about 45:1, about 50:1, about 55:1, about 60:1, about 65:1, about 70:1, about 75:1, about 80:1, about 85:1, about 90:1, about 95:1, or about 100:1. In some aspects, the oligonucleotides and phospholipids form an oligonucleotide-lipid complex, such as, for example, a liposome complex. In some aspects, at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the liposomes are less than 5 microns in diameter. In various aspects, the composition further comprises at least one surfactant, such as, for example, polysorbate 20. In some aspects, at least about 5% of the total liposomal antisense drug product consists of surfactant and at least about 90% of the liposomes are less than 5 microns in diameter. In some aspects, at least about 15% of the total liposomal antisense drug product consists of surfactant and at least about 90% of the liposomes are less than 3 microns in diameter. In some aspects, the population of oligonucleotides are incorporated in the population of liposomes.

In some aspects, the pharmaceutical composition is a liquid pharmaceutical composition. In other aspects, the pharmaceutical composition is a solid pharmaceutical composition.

Dosages for systemic administration of the antisense in human subjects may be about 0.025 g/kg, about 0.05 g/kg, about 0.1 g/kg, about 0.15 g/kg, or about 0.2 g/kg. In certain embodiments, the dosage for systemic administration may be from 0.025 g/kg to 0.2 g/kg. In some embodiments, the dosage is about 0.2 g/kg. In other embodiments, the dosage is from 0.004 g/kg to 0.01 g/kg. In other embodiments, the dosage is less than 0.01 g/kg. In further embodiments, the dosage is not between 0.01 g/kg to 0.2 g/kg. In certain aspects, the antisense is supplied as a lyophilized powder and re-suspended prior to administration. When resuspended the concentration of the antisense may be about 50 mg/ml, about 100 mg/ml, about 200 mg/ml, about 500 mg/ml, about 1000 mg/ml, or a range between those amounts.

In certain embodiments, the AS ODN may be administered systemically pre-operatively; for example prior to surgery to reduce tumor burden. For example, the AS ODN may be administered up to 24 hours, up to 36 hours, up to 48 hours or up to 72 hours before surgery. In particular aspects, the pharmaceutical composition may be administered about 48 to about 72 hours before surgery. Typically, in such circumstances, the administration is by intravenous bolus.

### Combination Therapies

Bladder cancer therapy has involved treating subjects with radiation, chemotherapy, biological therapies, hormone therapies, or combinations thereof. Such approaches have well-documented challenges. Advantageously, however, the chamber implantation methods disclosed herein may be used to treat a subject having cancer as a monotherapy. Thus it is preferable that the methods disclosed herein do not include chemotherapy, radiation therapy, biological therapy, and/or hormone therapy. Notwithstanding the excellent effect achieved by monotherapy approaches herein, however, it may be beneficial under certain circumstances to combine the chamber methods with other therapies; for example, radiation therapy. In certain embodiments, the radiation therapy includes, but is not limited to, internal source radiation therapy, external beam radiation therapy, and systemic radioisotope radiation therapy. In certain aspects, the radiation therapy is external beam radiation therapy. In some embodiments, the external beam radiation therapy includes, but is not limited to, gamma radiation therapy, X-ray therapy, intensity modulated radiation therapy (IMRT), and image-guided radiation therapy (IGRT). In certain embodiments, the external beam radiation therapy is gamma radiation therapy. Radiation may be administered before chamber implantation or after implantation; for example, as a salvage therapy. Typically, such salvage therapy approaches are not implemented until the cancer is determined to have returned.

Thus, in certain combination approaches, both the chamber methods, and the systemic methods and compositions, described herein may be used in the same subject, alone or in combination with one or more of radiation therapy, chemotherapy, hormone therapy, and biologic therapy. In the combination approaches described herein, the chamber implantation is preferably used as a first-line therapy. Using the chamber implantation first is desirable because the subject's immune system can be inhibited by other therapies, reducing the therapeutic benefit of the chamber implantation.

Optionally, systemic administration of the AS ODN may be performed prior to chamber implantation. Such an approach can be used to enhance the subjects immune system, as a priming approach. The priming approach may be especially advantageous where prior therapy has resulted in the subject having a compromised immune system.

When systemic administration is used in combination, the AS ODN may be systemically administered at least 2 weeks, at least 1 week, at least 3 days, or at least 1 day prior to treatment of the patient using an autologous cancer cell vaccine. In other embodiments, the AS ODN may be systemically administered at least 1 day, at least 3 days, at least 1 week, or at least 2 weeks following treatment of the patient using an autologous cancer cell vaccine; i.e. the chamber.

Optionally, the subject may be revaccinated with chambers using the methods described here subsequent to the first vaccination. A second or further additional vaccination may use tumor cells taken from the subject during the tissue removal and stored. Optionally, the second or further additional vaccination may use fresh tumor tissue removed from the subject and treated as described herein. Any tumor remaining in the subject may express the same antigens and thus act as a depot, providing for re-stimulation. However, recurring tumors may develop new antigens and thus provide additional options to stimulate an anti-tumor response. A subsequent vaccination may be after the first treatment is complete and the tumor has recurred or if the subject has not responded to the first treatment.

Subjects for Treatment with the IGF-1R as ODN

Suitable subjects are animals with bladder cancer; typically, the subject is a human. The disclosure provides methods of treating bladder cancer, including metastatic bladder cancer.

In some embodiments, the subject may have a gene mutation known to be associated with an increased risk of bladder cancer.

The subject is preferably one who has not been previously treated with any therapeutic approaches that are immunosuppressive.

Optionally, a subject who is a candidate for treatment may be identified by performing a tumor biopsy on the subject.

### Target Cells

Without being bound by any theory it is thought that the AS ODN reduces the subjects M2 cells and/or inhibits polarization of cells into M2 cells by downregulating IGF-1R expression. In some embodiments, IGF-1R expression in M2 cells is downregulated by at least about 1%, at least about 2%, at least about 5%, at least about 10%, at least about 20%, at least about 30%, at least about 40%, at least about 50%, at least about 60%, at least about 70%, at least about 80%, at least about 90%, or at least about 95% in comparison to cells not treated with the antisense. IGF-1R expression in M2 cells may be measured by quantitative RT-PCR.

In some embodiments, IGF-1R expression in M2 cells remains downregulated in the subject for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 3 weeks, at least about 4 weeks, at least about 5 weeks, or at least about 6 weeks after receiving one dose of the antisense.

In some aspects, the downregulation of expression of IGF-1R in M2 cells causes a selective reduction of M2 cells in a subject in comparison to cells not expressing IGF-1R. In certain embodiments, M2 cells in a subject are reduced by at least about 2%, at least about 5%, at least about 10%, at least about 20%, at least about 30%, at least about 40%, at least about 50%, at least about 60%, at least about 70%, at least about 80%, at least about 90%, or at least about 95% in comparison to a subject not treated with the antisense. In other embodiments, the M2 cell population is eliminated. For example, after implantation of the biodiffusion chamber, the M2 cell population may be about 1%, about 2%, about 5%, or about 10% of the population before implantation of the biodiffusion chamber. M2 cells in a subject may be measured using FACS. In certain aspects, after treatment the M2 cells are eliminated; i.e., undetectable by FACS. In other aspects, the decrease in M2 cells may be measured using a proxy assay; for example, serum from the subject may be obtained before and after treatment to assess its ability to polarize M2 cells. Following treatment with methods disclosed herein, the ability of the serum to polarize M2 cells is reduced by about 80% to about 100%, about 20% to about 60%, or about 10% to about 50%.

In some embodiments, targeting the expression of IGF-1R in M2 cells causes the M2 cells to undergo cell death. In certain embodiments, the cell death is necrosis. In other embodiments, the cell death is apoptosis. Apoptosis, for purposes of this disclosure, is defined as programmed cell death and includes, but is not limited to, regression of primary and metastatic tumors. Apoptosis is a programmed cell death which is a widespread phenomenon that plays a crucial role in the myriad of physiological and pathological processes. Necrosis, in contrast, is an accidental cell death which is the cell's response to a variety of harmful conditions and toxic substances. In yet other embodiments, targeting the expression of IGF-1R in M2 cells causes the M2 cells to undergo cell cycle arrest.

### Kits

Preparation of a completed chamber requires multiple components and multiple steps. In another aspects of the disclosure kits containing components for practicing the methods disclosed herein are provided. In certain aspects, the kits comprise the chamber body, which may be present in one portion or in two halves. Items to seal the chamber may also be included including one or more membranes, glues and solvents (e.g., an alcohol, or 2 dichloroethane). Optionally, the membrane may by sonically welded onto the chamber to create a seal. The kits include the antisense ODN. Optionally, the ODN may be divided into two portions. A first portion to treat the cells after surgical removal from the subject, and a second portion to combine with the cells when introduced into the subject. Other optional kit items include media for culturing the cells, and antibiotics for preventing bacterial growth in the media.

Optionally, chambers in the kit may be pre-connected (e.g by suture) to each other using an eyelet or other device attached to the chamber and adapted to receive the connecting material. Advantageously, by pre-connecting multiple chambers, the desired number of chambers may be readily introduced and removed by the surgeon.

### Numbered Embodiments of the Invention

In addition to the aspects and embodiments contemplated elsewhere in the instant disclosure, the following numbered embodiments are specifically contemplated:
1. A method for preparing a biodiffusion chamber for implantation into a subject having bladder cancer, the method comprising:
   (a) encapsulating tumor cells obtained from the subject into the biodiffusion chamber; and
   (b) irradiating the biodiffusion chamber.
2. A method for preparing a biodiffusion chamber for implantation into a subject having bladder cancer, the method comprising:
   (a) encapsulating tumor cells obtained from the subject into the biodiffusion chamber in the presence of an IGF-1R AS ODN; and
   (b) irradiating the biodiffusion chamber.
3. A biodiffusion chamber for implantation into a subject having bladder cancer, the biodiffusion chamber comprising:
   (a) irradiated tumor cells,
   wherein the tumor cells comprise adherent cells obtained from the subject's tumor tissue.
4. A biodiffusion chamber for implantation into a subject having bladder cancer, the biodiffusion chamber comprising:
   (a) irradiated tumor cells,
   wherein the tumor cells comprise adherent cells obtained from the subject's tumor tissue;
   wherein the tumor cells are pre-incubated with insulin-like growth factor receptor-1 antisense oligodeoxynucleotide (IGF-1R AS ODN) prior to encapsulation within the chamber; and
   (b) irradiated IGF-1R AS ODN wherein the IGF-1R AS ODN has the sequence of SEQ ID NO: 1.
5. A method of vaccinating a subject having bladder cancer comprising:
   (i) obtaining tumor tissue from the subject;
   (ii) harvesting adherent cells from the tissue;
   (iii) encapsulating the harvested cells in a biodiffusion chamber;
   (iv) irradiating the chamber, and
   (v) implanting the chamber in the subject,
wherein an immune response against the bladder cancer is obtained.
6. A method of vaccinating a subject having bladder cancer comprising:
   (i) obtaining a tumor tissue from the subject;
   (ii) collecting the tumor tissue in a sterile trap;
   (iii) harvesting adherent cells from the tumor tissue;
   (iv) encapsulating the harvested cells in a biodiffusion chamber along with insulin-like growth factor receptor-1 antisense oligodeoxynucleotide (IGF-1R AS ODN) having the sequence of SEQ ID NO:1;
   (v) irradiating the chamber, and
   (vi) implanting the chamber in the subject,
wherein an immune response against the bladder cancer is obtained.
7. The method or composition of any of the preceding embodiments, wherein the bladder cancer is a locally advanced or metastatic urothelial cancer (mUC).
8. The method or composition of any of the preceding embodiments, wherein the bladder cancer is muscle invasive urothelial carcinoma.
9 The method or composition of any of the preceding embodiments, wherein the bladder cancer is non-muscle invasive urothelial carcinoma.
10. The method or composition of any of the preceding embodiments, wherein the bladder cancer is Bacillus Calmette-Guerin (BCG)-unresponsive, high-risk, non-muscle invasive bladder cancer (NMIBC) with carcinoma in situ (CIS) with or without papillary tumors in a patient is ineligible for or has elected not to undergo cystectomy.
11. The method or composition of any of the preceding embodiments, wherein the bladder cancer is upper tract urothelial cancer.
12. The method or composition of any of the preceding embodiments, wherein the bladder cancer is low grade upper tract urothelial cancer (LG-UTUC).
13. The method or composition of any one of the preceding embodiments wherein the dose of radiation is between 1 Gy and 15 Gy.
14. The method or composition of any one of the preceding embodiments wherein the dose of radiation is about 1 Gy.
15. The method or composition of any one of the preceding embodiments wherein the dose of radiation is about 2 Gy.
16. The method or composition of any one of the preceding embodiments wherein the dose of radiation is about 3 Gy.
17. The method or composition of any one of the preceding embodiments wherein the dose of radiation is about 4 Gy.
18. The method or composition of any one of the preceding embodiments wherein the dose of radiation is about 5 Gy.
19. The method or composition of any of the preceding embodiments, wherein the tumor cells are dispersed before adding to the chamber.
20. The method or composition of any of the preceding embodiments, wherein the cells are not exposed to temperatures above body temperature during removal from the subject.
21. The method or composition of any of the preceding embodiments, wherein the cells are not exposed to temperatures above 37 °C during removal from the subject.
22. The method or composition of any of the preceding embodiments, wherein the tumor tissues is removed from the subject using a tissue morselator comprising a sterile trap.
23. The method or composition of any of the preceding embodiments, wherein the tumor tissues is removed from the subject using a tissue morselator that comprises a high-speed reciprocating inner cannula within a stationary outer cannula.
24. The method or composition of any of the preceding embodiments, wherein the tumor cells are enriched for nestin expression before they are placed into the biodiffusion chamber.
25. The method or composition of any of the preceding embodiments, wherein the tumor cells in the chamber are enriched for adherent cells compared to the tumor cells obtained from the subject.
26. The method or composition of any of the preceding embodiments, wherein the tumor cells consist essentially of adherent cells.
27. The method or composition of any of the preceding embodiments, wherein the cells are treated with IGF-1R AS ODN before encapsulation into the chamber.
28. The method of any of the preceding embodiments, wherein the IGF-1R AS ODN is present at about 2 mg to about 6 mg per million cells during the treatment before encapsulation.
29. The method or composition of any of the preceding embodiments, wherein the IGF-1R AS ODN is present at about 4 mg per million cells during the treatment before encapsulation.
30 The method or composition of any of the preceding embodiments, wherein the treatment with IGF-1R AS ODN prior to encapsulation is for up to about 18 hours.
31. The method or composition of any of the preceding embodiments, wherein the treatment with IGF-1R AS ODN prior to encapsulation is for about 12 hours to about 18 hours.
32. The method or composition of any of the preceding embodiments, wherein the IGF-1R AS ODN has the sequence of SEQ ID NO:1.
33. A method of treating a subject having bladder cancer, comprising implanting two or more biodiffusion chambers according to any one of the preceding embodiments into the subject.
34. The method or composition of any of the preceding embodiments, wherein about 10 to about 30 biodiffusion chambers are implanted into the subject.
35. The method or composition of any of the preceding embodiments, wherein about 10 to about 20 biodiffusion chambers are implanted into the subject.
36. The method or composition of any of the preceding embodiments, wherein the diffusion chambers are implanted into the subject for 48 hours.
37 The method or composition of any of the preceding embodiments, wherein the method is performed without chemotherapy, without radiation therapy, or without both.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out certain embodiments of the present invention, and are not to be construed as limiting in any way the remainder of the disclosure.

### Example 1—Bladder Cancer Vaccination

To test the effectiveness of the general cancer vaccination platform described in D. Andrews, K. Judy, C. Scott, S. Garcia, L. Harshyn, L. Kenyon, K. Talekar, A. Flanders, K. Atsina, L. Kim, N. Martinez, W. Shi, M. Werner-Wasik, H. Liu, M. Prosniak, M. Curtis, R. Kean, D. Ye, E. Bongiorno, S. Sauma, M. Exley, K. Pigott and D. Hooper, “Phase Ib Clinical Trial of IGV-001 for Patients with Newly Diagnosed Glioblastoma,” Clin Cancer Res, vol. 27, no. 7, pp. 1912-1922, 2021 orthotopic MBT-2 urothelial murine cancer model was used. BDCs containing saline or 1×10⁶ IMV-001-treated tumor cells (IUC-001 respectively) were implanted in flanks of mice and explanted 48 h later, as per glioblastoma clinical protocol. Primary tumor challenge was conducted 28d after chamber implantation. Mice were monitored for survival and tumor growth. Cytokine analysis and immunophenotyping were conducted.

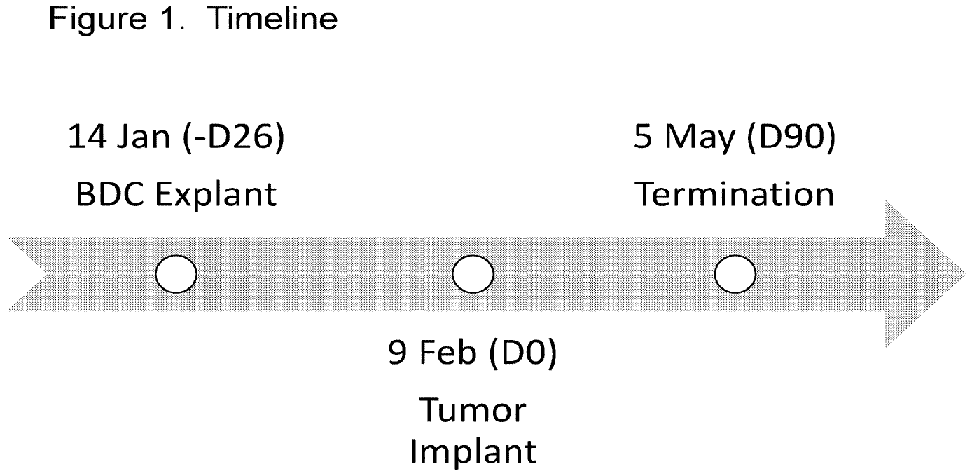

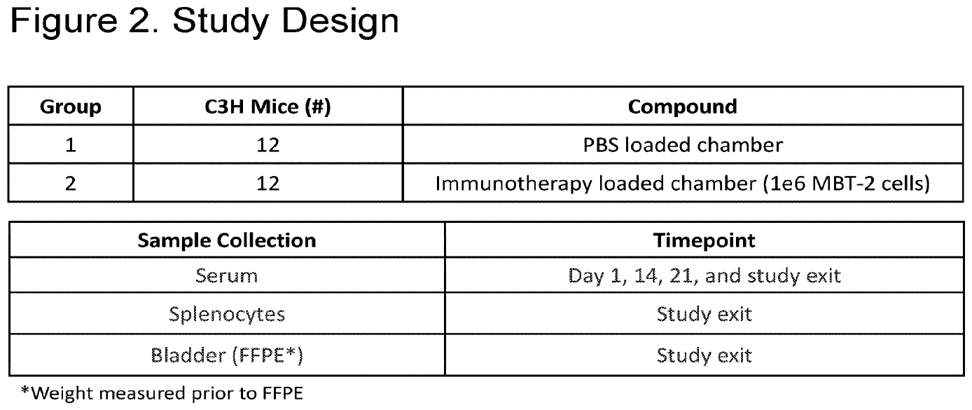

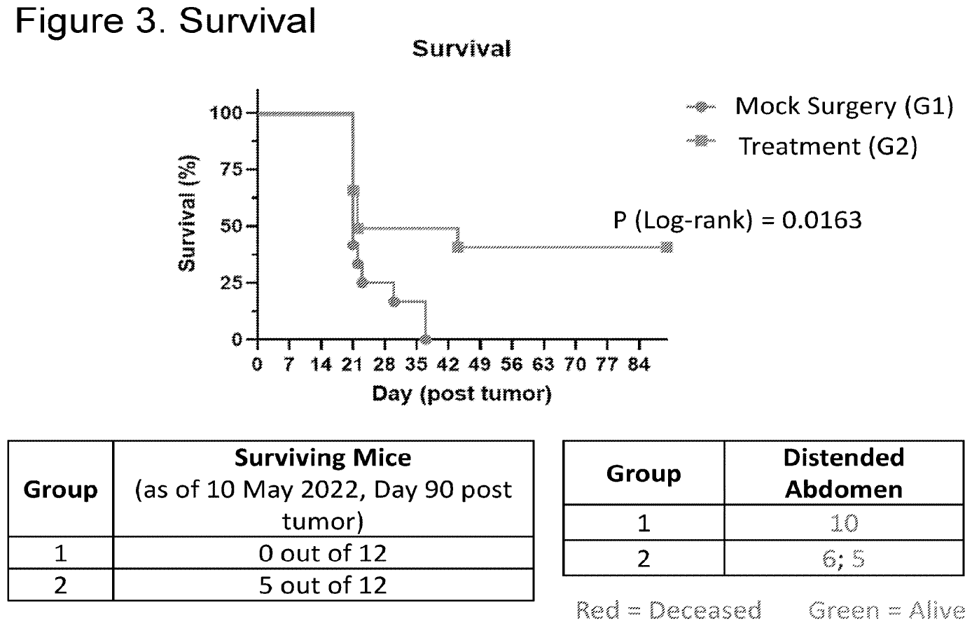

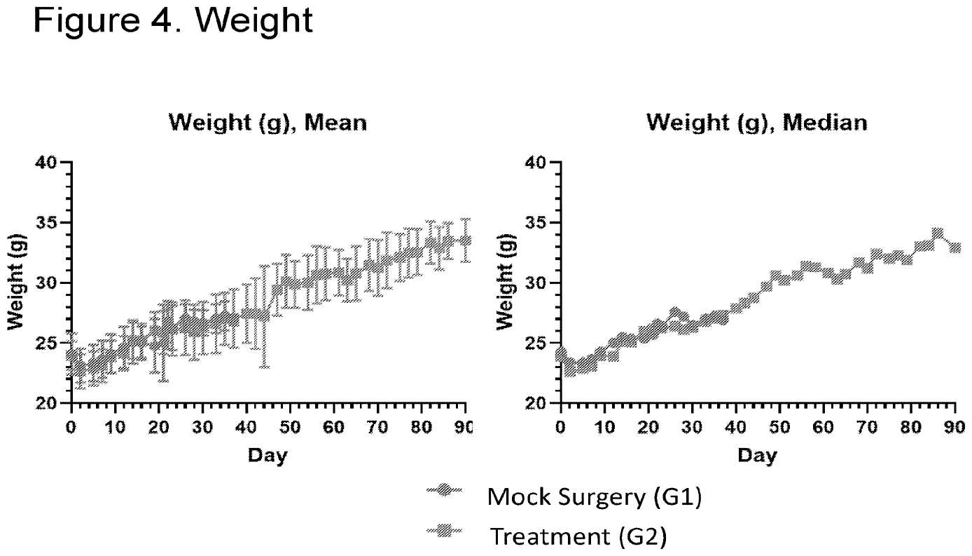

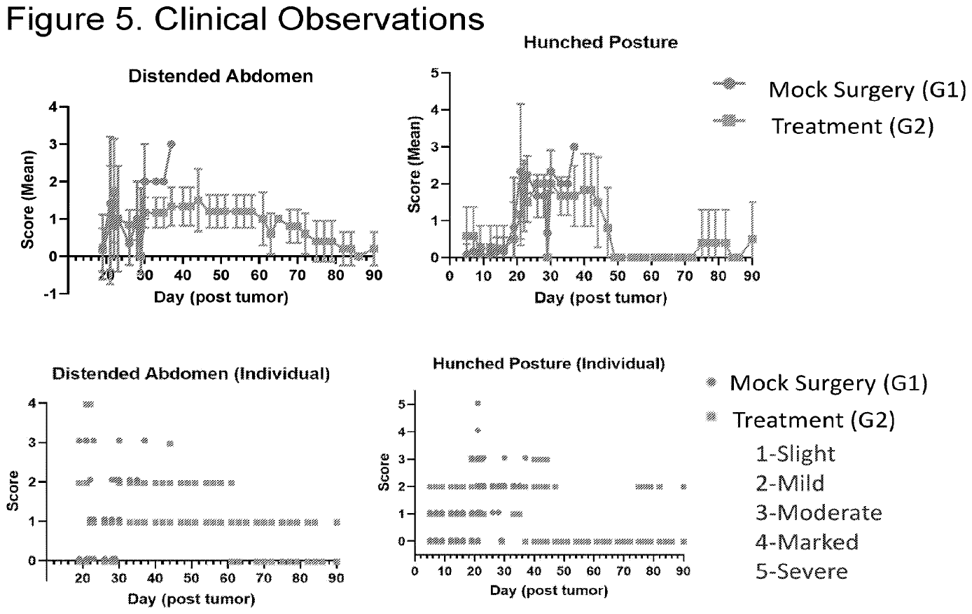

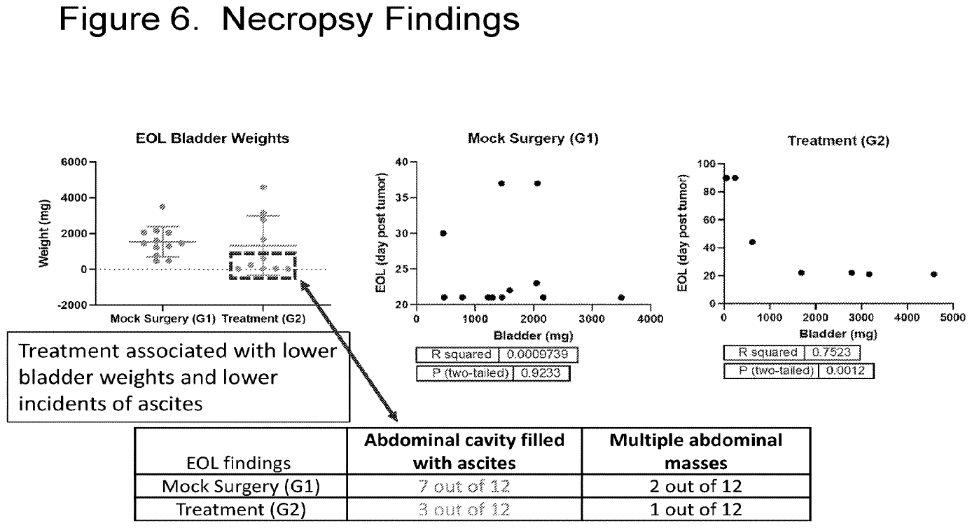

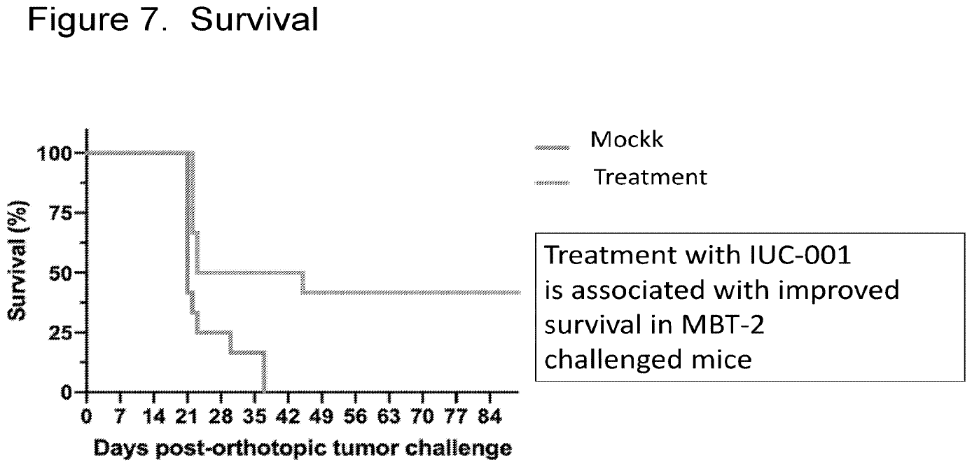

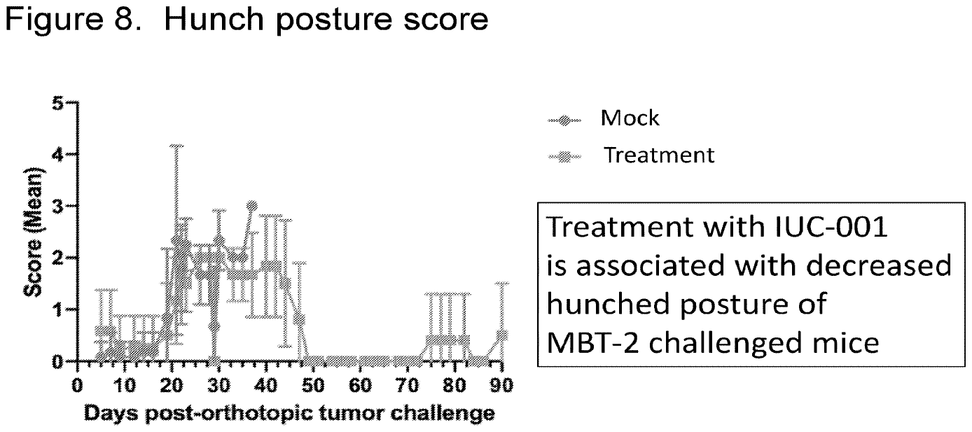

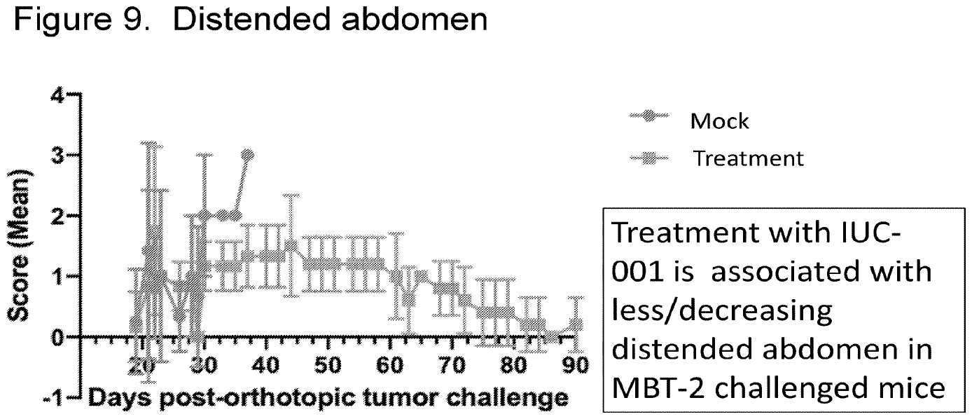

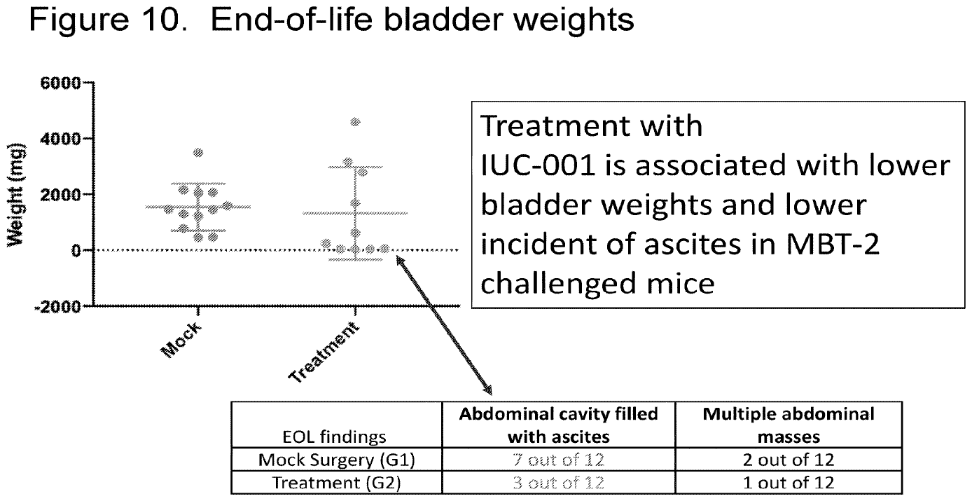

In the MBT-2 model, 42% of IUC-001-treated mice survived up to 90d post-tumor challenge (MST=33d at end of study); all control mice expired by 37d (MST=21d; P=0.0163). Low end of life bladder weights in surviving IUC-001-treated mice suggested minimal tumor burden. Additional protocol and results information from this study are shown in FIGS. 1-10. These data support the durable antitumor activity of the immunotherapeutic platform in urothelial cancer. Results suggest that efficacy is associated with a systemic immunological response, resulting in generation of Th1 antitumor cytotoxic T cells.

### Example 2—Vaccination of a Human Patient Suffering from Bladder Cancer

Tumor tissue is removed from the bladder of bladder cancer patients and then transferred to a designated BSL-2 facility, where the tumor tissue is processed and placed into biodiffusion chambers.

The biodiffusion chambers contain autologous tumor cells removed at surgery. Prior to being added to the biodiffusion chambers, the cells are pretreated overnight (approx. 12-18 hours) with a first amount (4 mg/ml) of an 18-mer IGF-1R AS ODN with the sequence 5′-TCCTCCGGAGCCAGACTT-3′ (NOBEL) (SEQ ID NO: 1). Based on data showing that the AS ODN has immunomodulatory properties, a second amount (2 μg) of exogenous NOBEL antisense is added to the chambers (C-v), and the chambers are subsequently irradiated. The ratio of tumor cells to IGF-1R AS ODN in the chamber is in a range from about 3.75×10⁵:1 μg to about 6.25×10⁵:1 μg.

Bladder cancer patients are identified for treatment before, after, or concurrently with treatment using standard therapy. Each patient may meet the following criteria: age >18, and no co-morbidities that would preclude treatment.

Biodiffusion chambers are prepared as described above. The day following surgery to remove tumor tissue, 2-20 irradiated biodiffusion chambers are implanted into the rectus sheath of the patients. After 24-48 hours, the chambers are removed. Optionally, an antisense nucleotide against IGFR-1 is administered concurrently.

Clinical efficacy is monitored over time using appropriate bladder cancer assessments.

Treatment may be repeated as necessary to inhibit and/or eliminate tumor growth.

### INCORPORATION BY REFERENCE

All patents and publications referenced herein are hereby incorporated by reference in their entireties for all purposes.

What is claimed is:

1. A method for preparing a biodiffusion chamber for implantation into a subject having bladder cancer, the method comprising:
(a) encapsulating tumor cells obtained from the subject into the biodiffusion chamber; and
(b) irradiating the biodiffusion chamber.

2. A method for preparing a biodiffusion chamber for implantation into a subject having bladder cancer, the method comprising:
(a) encapsulating tumor cells obtained from the subject into the biodiffusion chamber in the presence of an IGF-1R AS ODN; and
(b) irradiating the biodiffusion chamber.

3. A biodiffusion chamber for implantation into a subject having bladder cancer, the biodiffusion chamber comprising:
(a) irradiated tumor cells,
wherein the tumor cells comprise adherent cells obtained from the subject's tumor tissue.

4. A biodiffusion chamber for implantation into a subject having bladder cancer, the biodiffusion chamber comprising:
(a) irradiated tumor cells,
wherein the tumor cells comprise adherent cells obtained from the subject's tumor tissue;
wherein the tumor cells are pre-incubated with insulin-like growth factor receptor-1 antisense oligodeoxynucleotide (IGF-1R AS ODN) prior to encapsulation within the chamber; and
(b) irradiated IGF-1R AS ODN wherein the IGF-1R AS ODN has the sequence of SEQ ID NO: 1.

5. A method of vaccinating a subject having bladder cancer comprising:
(i) obtaining tumor tissue from the subject;
(ii) harvesting adherent cells from the tissue;
(iii) encapsulating the harvested cells in a biodiffusion chamber;
(iv) irradiating the chamber, and
(v) implanting the chamber in the subject,
wherein an immune response against the bladder cancer is obtained.

6. A method of vaccinating a subject having bladder cancer comprising:
(i) obtaining a tumor tissue from the subject;
(ii) collecting the tumor tissue in a sterile trap;
(iii) harvesting adherent cells from the tumor tissue;
(iv) encapsulating the harvested cells in a biodiffusion chamber along with insulin-like growth factor receptor-1 antisense oligodeoxynucleotide (IGF-1R AS ODN) having the sequence of SEQ ID NO:1;
(v) irradiating the chamber, and
(vi) implanting the chamber in the subject,
wherein an immune response against the bladder cancer is obtained.

7. The method or composition of any of the preceding claims, wherein the bladder cancer is a locally advanced or metastatic urothelial cancer (mUC).

8. The method or composition of any of the preceding claims, wherein the bladder cancer is muscle invasive urothelial carcinoma.

9. The method or composition of any of the preceding claims, wherein the bladder cancer is non-muscle invasive urothelial carcinoma.

10. The method or composition of any of the preceding claims, wherein the bladder cancer is Bacillus Calmette-Guerin (BCG)-unresponsive, high-risk, non-muscle invasive bladder cancer (NMIBC) with carcinoma in situ (CIS) with or without papillary tumors in a patient is ineligible for or has elected not to undergo cystectomy.

11. The method or composition of any of the preceding claims, wherein the bladder cancer is upper tract urothelial cancer.

12. The method or composition of any of the preceding claims, wherein the bladder cancer is low grade upper tract urothelial cancer (LG-UTUC).

13. The method or composition of any one of the preceding claims wherein the dose of radiation is between 1 Gy and 15 Gy.

14. The method or composition of any one of the preceding claims wherein the dose of radiation is about 1 Gy.

15. The method or composition of any one of the preceding claims wherein the dose of radiation is about 2 Gy.

16. The method or composition of any one of the preceding claims wherein the dose of radiation is about 3 Gy.

17. The method or composition of any one of the preceding claims wherein the dose of radiation is about 4 Gy.

18. The method or composition of any one of the preceding claims wherein the dose of radiation is about 5 Gy.

19. The method or composition of any of the preceding claims, wherein the tumor cells are dispersed before adding to the chamber.

20. The method or composition of any of the preceding claims, wherein the cells are not exposed to temperatures above body temperature during removal from the subject.

21. The method or composition of any of the preceding claims, wherein the cells are not exposed to temperatures above 37 °C during removal from the subject.

22. The method or composition of any of the preceding claims, wherein the tumor tissues is removed from the subject using a tissue morselator comprising a sterile trap.

23. The method or composition of any of the preceding claims, wherein the tumor tissues is removed from the subject using a tissue morselator that comprises a high-speed reciprocating inner cannula within a stationary outer cannula.

24. The method or composition of any of the preceding claims, wherein the tumor cells are enriched for nestin expression before they are placed into the biodiffusion chamber.

25. The method or composition of any of the preceding claims, wherein the tumor cells in the chamber are enriched for adherent cells compared to the tumor cells obtained from the subject.

26. The method or composition of any of the preceding claims, wherein the tumor cells consist essentially of adherent cells.

27. The method or composition of any of the preceding claims, wherein the cells are treated with IGF-1R AS ODN before encapsulation into the chamber.

28. The method of any of the preceding claims, wherein the IGF-1R AS ODN is present at about 2 mg to about 6 mg per million cells during the treatment before encapsulation.

29. The method or composition of any of the preceding claims, wherein the IGF-1R AS ODN is present at about 4 mg per million cells during the treatment before encapsulation.

30. The method or composition of any of the preceding claims, wherein the treatment with IGF-1R AS ODN prior to encapsulation is for up to about 18 hours.

31. The method or composition of any of the preceding claims, wherein the treatment with IGF-1R AS ODN prior to encapsulation is for about 12 hours to about 18 hours.

32. The method or composition of any of the preceding claims, wherein the IGF-1R AS ODN has the sequence of SEQ ID NO:1.

33. A method of treating a subject having bladder cancer, comprising implanting two or more biodiffusion chambers according to any one of the preceding claims into the subject.

34. The method or composition of any of the preceding claims, wherein about 10 to about 30 biodiffusion chambers are implanted into the subject.

35. The method or composition of any of the preceding claims, wherein about 10 to about 20 biodiffusion chambers are implanted into the subject.

36. The method or composition of any of the preceding claims, wherein the diffusion chambers are implanted into the subject for 48 hours.

37. The method or composition of any of the preceding claims, wherein the method is performed without chemotherapy, without radiation therapy, or without both.
